(19) United States
(12) Patent Application Publication
(10) Pub. No.: US 2003/0065276 A1
(43) Pub. Date: Apr. 3, 2003

(54) Audiometer

(75) Inventor: Junichi Akita, Gamagori (JP)

Correspondence Address: SUGHRUE MION, PLLC, 2100 PENNSYLVANIA AVENUE, N.W., WASHINGTON, DC, 20037, US

(73) Assignee: NIDEK CO., LTD.

(21) Appl. No.: 10/254,604

(22) Filed: Sep. 26, 2002

(30) Foreign Application Priority Data
Sep. 28, 2001 (JP) P2001-304312

(51) Int. Cl. A61B 5/00

(52) U.S. Cl. 600/559

(57) ABSTRACT

An audiometer includes: sound output means for externally outputting a test sound for testing hearing; sound input means for fetching the output test sound; storage means for storing a reference signal value for a predetermined sound pressure level of a predetermined frequency; detection means for detecting a sound pressure level signal value of the fetched test sound; instruction means for instructing a calibration start; and calibration means for, when the calibration start is instructed, permitting the sound output means to output the test sound based on the stored reference signal value, and for calibrating the output of the test sound so as to establish a predetermined relationship between the detected sound pressure level signal value and the reference signal value.

### BACKGROUND OF THE INVENTION

The present invention relates to an audiometer for testing the hearing acuity of a testee.

An audiometer is available that provides a test sound for a testee for testing his or her hearing acuity. For the audiometer, calibration is required periodically, or when the testing environment is changed, so as to maintain the accuracy of a test sound that is to be provided.

However, a technician whose specialty is the performance of calibrations is required, and an extended period of time is required because the calibration procedures are complicated.

### SUMMARY OF THE INVENTION

To resolve this conventional problem, it is one objective of the present invention to provide an audiometer that can be calibrated easily, without a technician whose specialty is the performance of calibrations being required.

The present invention provide, for example, the followings:

(1) An audiometer comprising:

sound output means for externally outputting a test sound for testing hearing;

sound input means for fetching the output test sound;

storage means for storing a reference signal value for a predetermined sound pressure level of a predetermined frequency;

detection means for detecting a sound pressure level signal value of the fetched test sound;

instruction means for instructing a calibration start; and

calibration means for, when the calibration start is instructed, permitting the sound output means to output the test sound based on the stored reference signal value, and for calibrating the output of the test sound so as to establish a predetermined relationship between the detected sound pressure level signal value and the reference signal value.

(2) An audiometer according to (1), wherein the calibration means compares the detected sound pressure level signal value with the reference signal value, and corrects the sound pressure level signal value to substantially match the reference signal value, to thereby calibrate the output of the test sound.

(3) An audiometer according to (1), wherein the storage means stores reference signal values for a predetermined sound pressure level of multiple types of predetermined frequencies,

the detection means performs frequency analysis of the fetched test sound, and detects the sound pressure level signal value for each of the frequencies, and

the calibration means permits the sound output means to simultaneously output the test sounds for multiple types of the frequencies, and calibrates the output of the test sound for each of the frequencies.

(4) An audiometer according to (1), wherein the storage means stores reference signal values for at least three predetermined sound pressure levels of predetermined frequency, and

the calibration means calibrates at least two types of sound pressure levels, and employs the calibration results to calibrate the sound pressure level of another type.

The present disclosure relates to the subject matter contained in Japanese patent application No. P2001-304312 (filed on Sep. 28, 2001), which is expressly incorporated herein by reference in its entirety.

### BRIEF DESCRIPTION OF THE DRAWINGS

FIG. 1 is a schematic diagram showing the external appearance of an audiometer.

FIG. 2 is a schematic block diagram showing the control system of the audiometer.

FIG. 3 is a flowchart showing the calibration process.

FIG. 4 is a flowchart showing the automatic hearing test process.

### DESCRIPTION OF THE PREFERRED EMBODIMENT

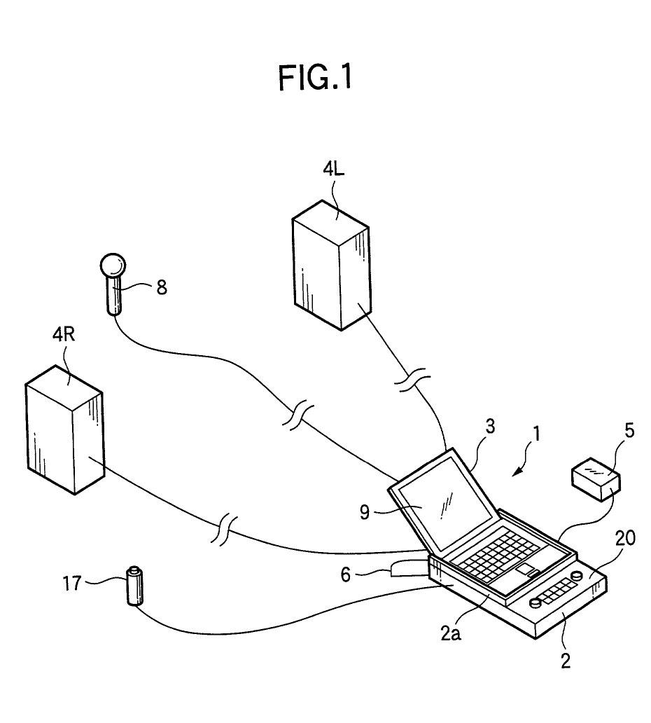

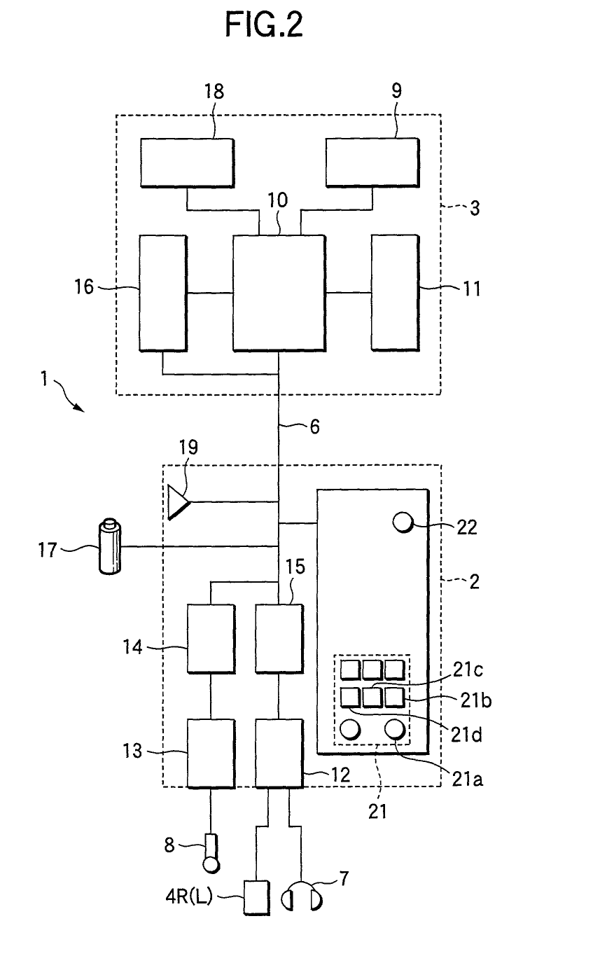

One embodiment of the present invention will now be described while referring to the drawings. FIG. 1 is a schematic diagram showing the external appearance of an audiometer according to the embodiment, and FIG. 2 is a schematic block diagram showing the control system of the audiometer.

A main body 1 for an audiometer comprises a base 2, on which a control panel 20 is mounted, used to designate various setups; and a notebook personal computer (hereinafter referred to as a PC) 3 mounted atop the base 2. A power source unit 5 is connected to the base 2, and as is shown in FIG. 2, the control panel 20 includes buttons 21 used to change (adjust) the sound pressure level, to select a frequency and to execute a testing program; and calibration button 22 used to calibrate the sound pressure level. The buttons 21 include a sound pressure change button 21a, used to enter a signal to change a sound pressure level; a start button 21b, used to enter a signal to start automatic test according to the testing program; a re-test button 21c, used to reenter a signal for the conduction of the test at the same sound pressure level; and a stop button 21d, used for entering a signal for halting of the automatic testing. It should be noted that, instead of using the control panel 20, a signal can be entered by using a mouse (not shown) to click on an operating screen on a display 9 of the PC 3.

Further, atop the base 2 an recessed portion 2a, the front edge (the control panel 20 side) and the right and left edges of which are raised, is formed into which an A4 size PC 3 can be fitted for mounting. Thus, after a PC 3 has been fitted into the recessed portion 2a atop the base 2, it is held securely while being operated. Although in this embodiment the top face of the base 2 is so designed that a PC 3 can be fitted therein, this is not the only shape that can be used for the base 2; any shape is acceptable so long as a PC 3 can be mounted on the top face. Furthermore, means for securing a PC 3 to the base 2 may be provided in order to prevent the PC 3 from separating from the base 2 during operation.

An output terminal 12, an input terminal 13 and a USB (Universal Serial Bus) terminal for the connection to the PC 3 are provided on the back face (rear face) of the base 2, and a USB cable 6 is used to connect the base 2 and the PC 3. While a conventional audiometer employs RS232C for a connection to a PC, in this embodiment, USB is employed, so that a connection can be effected while the PC 3 is on. In addition, since only the sound input/output units and the control panel 20 are mounted on the base 2, and since control of the main body 1 and the display of test results are tasks performed by the PC 3, the base 2 can be made compactly.

Output devices, such as a loudspeaker 4R (L) and a headphone 7, are connected to the output terminal 12, and an input device, such as a microphone 8 used for the calibration, is connected to the input terminal 13.

A control unit 10 provides control for the entire main body 1, and a memory 11 is used to store the testing program and reference signal values for test sounds. The reference signal values are numerical values for frequencies and sound pressure levels relative to each frequency. This numerical values are used as reference values for calibration. In this embodiment, seven different frequencies are used: 125 Hz, 250 Hz, 500 Hz, 1 KHz, 2 KHz, 4 KHz and 8 KHz, while the sound pressure levels for each frequency is −20 dB to 120 dB (either sequential or stepped application of the sound pressure levels may be used).

The control unit 10 transmits a reference signal value stored in the memory 11 as a digital signal to a D/A converter 15. The D/A converter 15 converts the digital signal into an analog signal that is output through the output terminal 12. Thereafter, a test sound is produced by the loudspeaker 4R (L) or the headphone 7 that is connected to the output terminal 12.

An A/D converter 14 is connected to the input terminal 13, and converts, into a digital signal, an analog signal for a test sound fetched (input) through the microphone 8. A signal detector 16 detects, from the digital signal obtained from the A/D converter 14, the frequency of the test sound and the output signal level (sound pressure level).

A response button 17 is used by a testee to enter a response, a memory 18 is used to store test results, and notification means 19 employs voice reproduction to provide test end notification.

The operation of the thus arranged audiometer will now be described. The calibration of the output of a test sound will be explained first, following which an explanation will be given for the automatic hearing test.

<Calibration of Output of a Test Sound>

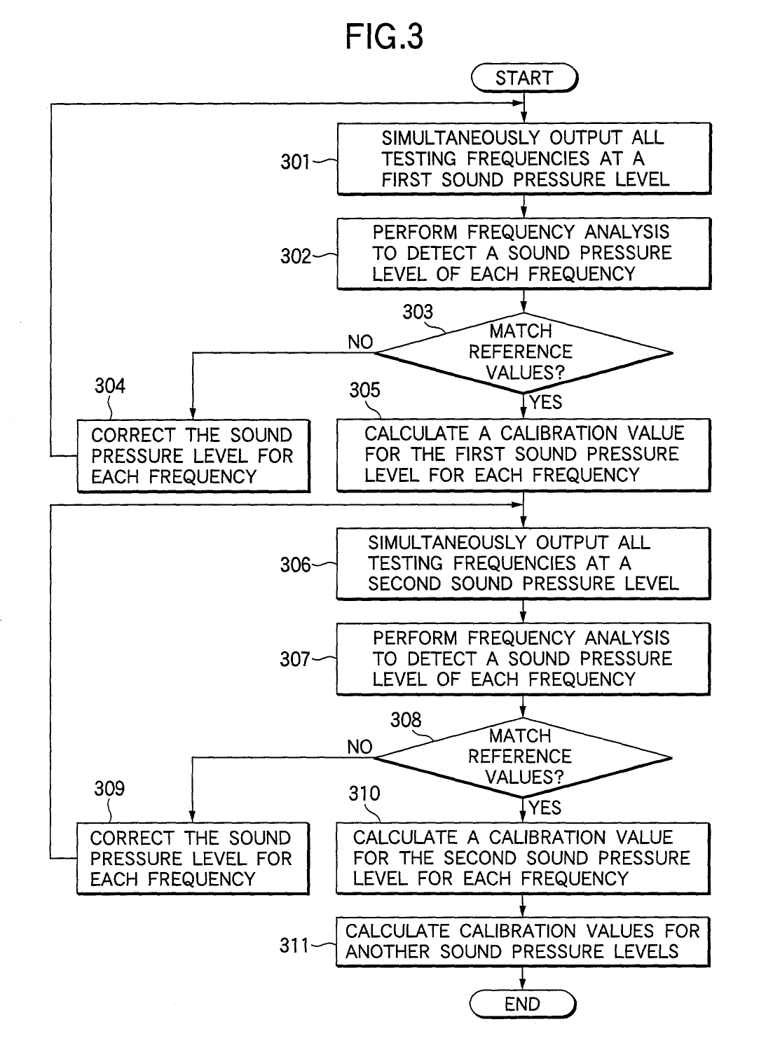

A calibration method employed when a loudspeaker is the sound output device that is used will be described while referring to the flowchart in FIG. 3.

First, a tester (the person responsible for performing the calibration) arranges the loudspeaker 4R (L) and the main body 1 at testing locations in accordance with a predetermined positional relationship, and sets up the microphone 8, which is connected to the input terminal 13, where it will be available for use by a testee. The microphone 8 has been calibrated in advance, and for the calibration, a correction value has been input to the main body 1 in advance.

Next, the tester employs the buttons 21 to select the loudspeaker 4 (R or L) to be calibrated. When the loudspeaker 4 has been selected, the tester depresses the button 22 to begin the calibration process.

Upon the depression of the button 22, the control unit 10 transmits to the D/A converter 15, as digital signals, reference signal values, which are selected from among all the reference signal values for a plurality of (all) frequencies and a predetermined (first) sound pressure level, e.g., 0 dB in this embodiment. The D/A converter 15 then converts the received digital signals into analog signals, and at the predetermined (first) sound pressure level, test sounds are produced by the loudspeaker 4R (L) for a plurality of (all) frequencies that are output at the same time through the output terminal 12 (301).

The test sounds released by the loudspeaker 4R (L) are picked up by the microphone 8, and are transmitted as analog signals through the input terminal 13 to the A/D converter 14, whereat the analog signals are converted into digital signals that are transmitted to the signal detector 16. The signal detector 16 performs computation, such as frequency analysis, for the received digital signals, and detects the sound pressure level signal values of the individual frequencies (302). Thereafter, the control unit 10 compares the obtained sound pressure level signal values of the frequencies with the corresponding reference signal values stored in the memory 11 to obtain differences in the values (303).

Subsequently, in order to match the reference signal values, the control unit 10 corrects the sound pressure level signal values by performing an arithmetic operation, such as addition or subtraction (304), and outputs through the loudspeaker 4R (L) the test sounds for which the sound pressure level signal values have been corrected (301). These test sounds are again fetched, compared and corrected (302-304). This comparison, correction and calibration process is repeated until the obtained sound pressure level signal values match the reference signal values stored in the memory 11 (305).

When the calibration process has been completed for the predetermined (first) sound pressure level, the control unit 10 performs the calibration process in the same manner for another predetermined (second) sound pressure level, e.g., 120 dB) (306-310). After the calibration process has been completed for the two types of sound pressure levels, the control unit 10 obtains a proportional relationship using the calibration values for the two sound pressure levels, and based on the proportional relationship, calculates calibration values for another sound pressure levels (the correction processes are performed) (311).

It should be noted that when there is a large difference between the two sound pressure levels output by the loudspeaker 4, the correction process for the calibration of another sound pressure level can be performed more accurately.

Conventionally, a technician whose specialty is the performance of calibrations must employ multiple external devices, such as sound level meters, and must spend an extended period of time to complete all the calibrations that are required. In this embodiment, however, the test sounds output by the main body 1 are fetched, the sound pressure level signal values of the test sounds are compared with the internally stored reference signal values, and the correction process is performed to match the values that are compared. Thus, calibrations can be easily performed without the services of a technician whose specialty is the performance of calibrations, and without external devices, such as sound level meters, being required.

Furthermore, since all the frequencies required for a hearing test are calibrated at the same time, compared with a method according to which frequencies are calibrated one by one, the time required for calibrations can be considerably reduced. Further, since to calibrate all the sound pressure levels only two different sound pressure levels must actually be output, the calibration time can be reduced even more.

While in this embodiment, all the frequencies required for a hearing test have been output at the same time at a predetermined sound pressure level, the individual frequencies may instead be output sequentially.

For the calibration process performed when the headphone 7 is employed, only a coupler (not shown) need be attached to the headphone 7, and the microphone 8 inserted therein. Then, using the calibration method previously described, the right and left sides of the headphone 7 can be calibrated.

<Hearing Test>

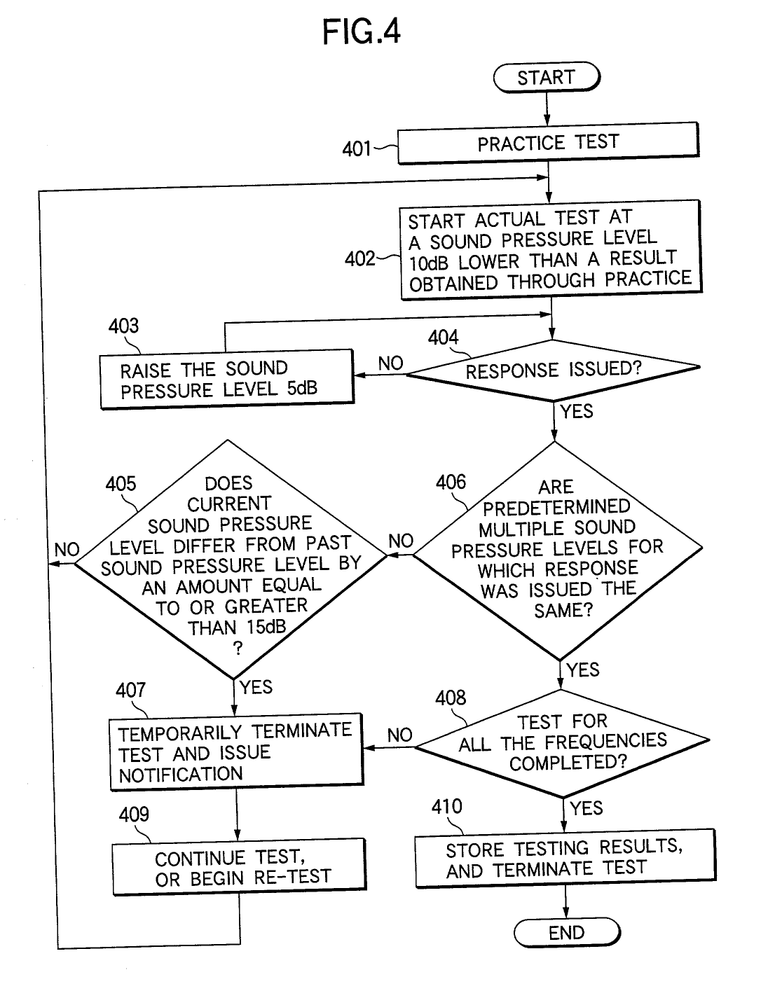

The hearing test will now be described while referring to the flowchart in FIG. 4. In this example, an explanation will be given for the automatic hearing test by which hearing ability is automatically tested using the headphone 7.

First, a tester permits a testee to hold the button 17 and attach the headphone 7 thereto. Then, the tester employs the button 21a to manually designate the approximate hearing ability of the testee (the threshold value of the testee's hearing ability for a specific frequency is designated using 10 dB units). Next, the tester uses the buttons 21 to store the obtained hearing ability in the memory 11 (or 18). Following this, the tester depresses the button 21b to begin the automatic hearing test, and the control unit 10 executes a testing program stored in the memory 11. It should be noted that the automatic hearing test is performed for each ear using the ascending method.

The control unit 10 first performs a practice hearing test. For this practice, at a frequency to be tested, such as 1 KHz, a sound pressure level that is lower (smaller) by 10 dB than the sound pressure level that was previously stored in the memory 11 is output by the headphone 7 for a specific period of time, and thereafter, the sound pressure level is raised (increased) by 5 dB. When the testee catches a sound produced by the headphone 7, he or she depresses the button 17 to notify the control unit 10 of the fact that he or she can identify the sound (401). From among a plurality of sound pressure levels presented for the practice, the control unit 10 determines, as a reference sound pressure, the minimum (lowest) sound pressure level that the testee can identify, and begins the actual hearing test. It should be noted that the testing program is broken down into a plurality of testing steps in accordance with the frequencies of the sounds that are to be presented.

At the first step, while employing 1 KHz as the frequency to be tested, the control unit 10 outputs, for a specific period of time, a sound pressure level that is lower (smaller) by 10 dB than the obtained reference sound pressure (402). When no response is received from the testee (404-N), the sound pressure level is raised (increased) 5 dB at a time until a response is received from the testee (403). Then, when a response is received (404-Y), the control unit 10 outputs the sound pressure level in the same manner, beginning with the level at the time of the start of the actual testing. The testing results are sequentially displayed on the display 9.

When a difference between predetermined multiple (e.g., two) sound pressure levels of which the response was issued is equal to or greater than 15 dB (405), it is assumed that the test may have been inappropriately conducted, and the control unit 10 temporarily halts the hearing test. Then, the control unit 10 permits the notification means 19 to generate a beep sound to notify the tester that the test has been halted temporarily (407). The tester then determines whether the test should be continued or be terminated. When the test is to be continued, the tester depresses the button 21c to perform hearing testing again at the 1 KHz frequency (409). When the test is to be terminated, the tester depresses the button 21d to terminate the hearing test, and performs the hearing test again from the beginning.

When the predetermined multiple (e.g., two) sound pressure levels of which the response was issued are the same (406), the control unit 10 stores, in the memory 18, the sound pressure level as the threshold value for the testee.

When the threshold value at 1 KHz for the testee is obtained, the control unit 10 terminates the test, and permits the notification means 19 to generate a beep sound to notify the tester that the test has been terminated temporarily. The tester then examines the testing results and the testing progress on the display 9 to determine whether the test was appropriate. When the tester determines that the test was appropriate, he or she depresses the button 21b to issue an instruction to the control unit 10 to perform hearing test for the next frequency (2 KHz). When the testing results are not satisfactory, the tester employs the button 21c to again perform the hearing test at 1 KHz.

When the testing steps have been conducted for all the frequencies that have been designated in advance (testing at 1 KHz, 2 KHz, 4 KHz, 8 KHz, 125 Hz, 250 Hz and 500 Hz is programmed) and the testing results have been obtained (408), the hearing test is ended (409). It should be noted that, even before all the testing steps have been completed, the testing program can also be halted by depressing the button 21d when a testing step has been completed.

Conventionally, once the automatic test has been started, the tester can not interrupt the operation until the test for all the frequencies has been completed. However, in this embodiment, since the operation is temporarily halted each time the test for one frequency has been completed, the tester can determine whether the test for each frequency has been appropriately performed.

In this embodiment, a notification has been issued by generating a sound; however, the notification may be issued visually by blinking the screen.

Furthermore, in this embodiment the test has been temporarily halted for each frequency. However, the buttons 21 may be employed to set an arbitrary interval for temporarily halting the test.

In addition, the appropriate testing state may be stored in advance in the memory 11, and the test may be temporarily halted and a notification issued only when inappropriate testing results are obtained.

As is described above, according to the present invention, the calibration can be easily performed without a technician whose specialty is the performance of calibrations.

What is claimed is:

1. An audiometer comprising:
sound output means for externally outputting a test sound for testing hearing;
sound input means for fetching the output test sound;
storage means for storing a reference signal value for a predetermined sound pressure level of a predetermined frequency;
detection means for detecting a sound pressure level signal value of the fetched test sound;
instruction means for instructing a calibration start; and
calibration means for, when the calibration start is instructed, permitting the sound output means to output the test sound based on the stored reference signal value, and for calibrating the output of the test sound so as to establish a predetermined relationship between the detected sound pressure level signal value and the reference signal value.

2. An audiometer according to claim 1, wherein the calibration means compares the detected sound pressure level signal value with the reference signal value, and corrects the sound pressure level signal value to substantially match the reference signal value, to thereby calibrate the output of the test sound.

3. An audiometer according to claim 1, wherein the storage means stores reference signal values for a predetermined sound pressure level of multiple types of predetermined frequencies,
the detection means performs frequency analysis of the fetched test sound, and detects the sound pressure level signal value for each of the frequencies, and
the calibration means permits the sound output means to simultaneously output the test sounds for multiple types of the frequencies, and calibrates the output of the test sound for each of the frequencies.

4. An audiometer according to claim 1, wherein the storage means stores reference signal values for at least three predetermined sound pressure levels of predetermined frequency, and
the calibration means calibrates at least two types of sound pressure levels, and employs the calibration results to calibrate the sound pressure level of another type.
